(19) United States
(12) Patent Application Publication
(10) Pub. No.: US 2015/0374747 A1
(43) Pub. Date: Dec. 31, 2015

(54) AMORPHOUS CALCIUM CARBONATE FOR ACCELERATED BONE GROWTH

(71) Applicant: AMORPHICAL LTD., Ness Ziona (IL)

(72) Inventors: Oren MEIRON, Beer Sheva (IL); Galit SHALTIEL-GOLD, Omer (IL); Michal DANIELY, Ganey-Tiqwa (IL)

(21) Appl. No.: 14/766,716

(22) PCT Filed: Feb. 10, 2014
(86) PCT No.: PCT/IL2014/050138

§ 371 Date: Aug. 7, 2015

Related U.S. Application Data
(60) Provisional application No. 61/762,981, filed on Feb. 11, 2013.

(51) Int. Cl. A61K 33/10; A61K 45/06; A61K 47/12; A61K 47/36; A61K 47/26; A61K 9/00; A61K 47/24

(52) U.S. Cl. CPC A61K 33/10; A61K 9/0053; A61K 45/06; A61K 47/24; A61K 47/36; A61K 47/26; A61K 47/12

(57) ABSTRACT

The present invention provides a method for accelerating bone growth in a subject having a bone condition, selected from the group consisting of a fracture by external force, pathological fracture, fatigue fracture, distraction osteogenesis, osteotomy, osseointegration and combinations thereof, the method employing the administration of a composition containing stable amorphous calcium carbonate, comprising at least one stabilizer. Further provided are the orally-administrable pharmaceutical compositions for use in accelerating bone growth in said bone conditions.

### FIELD OF THE INVENTION

The present invention relates to compositions and methods for acceleration of bone growth and bone fracture repair. The orally-administrable compositions of the present invention include stable amorphous calcium carbonate comprising at least one stabilizer. In particular, the compositions are useful for treating surgically induced fractures.

### BACKGROUND

A bone fracture is a medical condition in which there is a break in the continuity of the bone. A bone fracture can be the result of high force impact or stress or trivial injury as a result of certain medical conditions that weaken the bones, such as osteoporosis or bone cancer. Bones can also fracture as a result of repeated small stresses or strains. This type of fracture is known as a fatigue fracture and is common in athletes.

A bone fracture may be performed intentionally, for example by a bone dissection during an orthopedic surgery. Several surgeries including bone dissection are widely-used. For example, osteoclasis is an intentional surgical fracture of bone performed to correct deformity, e.g. to straighten a bone that has healed crookedly following a fracture. Osteotomy is a surgical operation whereby a bone is cut to shorten, lengthen, or change its alignment. It is performed to correct musculoskeletal deformities, such as coxa vara, genu valgum, and genu varum, as well as dentofacial deformities. Osteotomy is also a part of a distraction osteogenesis surgery, which involves bone dissection, distraction and a further consolidation of the elongated bone.

Fracture repair involves complex processes of cell and tissue proliferation and differentiation. Many players are involved, including growth factors, inflammatory cytokines, antioxidants, bone breakdown (osteoclast) and bone building (osteoblast) cells, hormones, amino acids, and uncounted nutrients.

The natural healing process of a fracture can be divided into three phases:
1. The inflammation phase—Immediately upon fracture, a blood clot forms, allowing the influx of inflammatory, clean-up cells to the wound area. This is followed by a cytokine cascade that brings the repair cells into the fracture gap. These cells immediately begin to differentiate into specialized cells that build new bone tissue (osteoblasts) and new cartilage (chondroblasts). Over the next few months, these cells begin the repair process, laying down new bone matrix and cartilage. At this initial stage, osteoclast cells dissolve and recycle bone debris. The process of laying down new bone material by osteoblasts is called osteogenesis or ossification.
2. The reparative stage—This process begins about two weeks after the fracture occurs. In this stage, proteins produced by the osteoblasts and chondroblasts begin to consolidate into what is known as a soft callus. This soft, new bone substance eventually hardens into a hard callus as the bone weaves together over a 6 to 12 week time period.
3. The remodeling phase—At this stage the callus begins to mature and remodel itself. Woven bone is remodeled into stronger lamellar bone by the orchestrated action of both osteoblast bone formation cells and osteoclast bone resorption cells.

Another biological process involving osteogenesis is osseointegration. Osseointegration refers to the direct structural and functional connection between living bone and the surface of a load-bearing artificial implant. Osseointegration procedure is a highly predictable and commonplace treatment modality, widely used in orthopedic and dental applications. Most commonly osseointegration procedure is used in the field of dental implants. Additional applications of osseointegration process include anchoring of orthopedic or craniofacial prosthesis and knee or joint replacement. Osseointegration surgery is performed in two stages, including a long waiting time after each of the stages. The total osseointegration process typically takes 12 months, although in some patients with poor bone quality a longer total rehabilitation time of up to 18 months is required.

Several types of population are susceptible to diminished bone healing. The use of several medicants, such as corticosteroids, chemotherapeutic agents, non-steroidal anti-inflammatory drugs, antibiotics, anticoagulants and drugs which reduce osteoclastic activity have been shown to affect bone healing [I. Pountos et at, Injury 2008 39:384-394]. An adequate blood supply is also mandatory for fracture healing, so that processes diminishing blood flow, such as smoking, slow the healing process. Fracture healing processes are also affected by the diabetic condition [D. T. Grave et at., JECM, 2011 3 (1):3-8]. Furthermore, elderly patients are susceptible to the impaired fracture healing [R. Gruber et al., Exp Gerontol. 2006 41(11):1080-93].

Bone healing is a natural process. Fracture treatment aims to ensure the best possible function of the injured part after healing. Treatment of bone fractures are broadly classified as surgical or conservative. Bone fractures are typically treated by restoring the fractured pieces of bone to their natural positions (if necessary), and maintaining those positions while the bone heals. A fractured limb is usually immobilized with a plaster or fiberglass cast or splint which holds the bones in position and immobilizes the joints above and below the fracture Smaller bones, such as fingers, are sometimes immobilized by bandaging them to a healthy adjacent finger, which serves a similar function to making a cast.

Only numerous studies attempted to test active treatments directed to stimulation of healing at the fracture site in cases of the bone fractures and bone dissection. Methods such as pulsed electromagnetic field devices, ultrasound (Low frequency devices) and injectable growth factors were described in several publications [Kristiansen et al., J Bone Joint Surg Am. 1997 79(7):961-973]. For example, U.S. Pat. No. 7,943,573 is directed to a method of stimulating osteogenesis during and/or following bone distraction, comprising providing a composition comprising a PDGF (platelet-derived growth factor) solution disposed in a biocompatible matrix and applying the composition to at least one site of bone distraction. Also, medications (injections of parathyroid hormone—PTH) can accelerate fracture healing with only limited success [Aspenberg et al., J Bone Miner. Res. 2010 25(2): 404-414; Aspenberg and Johansson Acta Orthop. 2010 81(2):234-236].

Similar techniques were also reported to promote bone growth in the osseointegration procedures. For example, it was shown that pulsed electro-magnetic stimulation may be useful for promoting bone formation around rough-surfaced dental implants [Matsumoto H, et al., Clin Oral Impl Res 2000: 11: 354-360]. In another study, effect of hypoxia-inducible factor-1α (HIF-1α) on bone regeneration and enhancing osseointegration in dental implants was evaluated [Zou D, He J, Zhang K, Dai J, Zhang W, et al. (2012) PLoS ONE 7(3): e32355]

The inorganic component of bone tissue, consisting of up to 50% of bone mass, is hydroxyapatite, a mineral form of calcium phosphate which is deposited by osteoblasts. Dietary supplements combining calcium and vitamin D₃ are sometimes prescribed after a fracture, however, there is no evidence that calcium supplementation by itself contributes to faster healing of a bone fracture.

Calcium is considered to be one of the most important minerals in the human body. It is required for maintaining bone mass, is essential for exocytosis of neurotransmitters, takes part in the contraction of muscle cells, replaces sodium as the depolarizing mineral in the heart, and participates in many other physiological functions.

Over the past 20 years, a rapidly growing scientific interest in the thermodynamically unstable amorphous polymorph of calcium carbonate, named amorphous calcium carbonate (ACC), has emerged. In nature, ACC is utilized by a small number of organisms, mainly crustaceans and other invertebrates that developed capabilities for stabilizing ACC in transient mineral deposition sites. These organisms require an exceptional efficient mineral source for the periodical mobilization, absorption and precipitation of calcium. In some crustaceans, such as the freshwater crayfish, ACC is stored in large quantities in specialized transient storage organs, named the Gastrolith.

International Patent Application No. WO 2005/115414 is directed to pharmaceutical and nutraceutical calcium compositions comprising gastrolith organs, ground to a fine powder. It was further disclosed that daily oral consumption of compositions comprising gastrolith components dramatically improves a range of conditions such as bone disorders, bone fractures, and cancer (WO 2008/041236).

International Patent Application No. WO 2009/053967 relates to pharmaceutical and nutraceutical compositions comprising ACC and phosphorylated peptides or amino acids for treating various disorders and conditions.

There still remains an unmet need for a method of accelerating healing of the fracture site in cases of the bone fractures and bone surgical dissection, and promoting bone growth in the osseointegration procedures, comprising administering an orally-administrable composition.

### SUMMARY OF THE INVENTION

The invention relates to compositions comprising stable amorphous calcium carbonate for rapidly and effectively promoting bone growth. The compositions of the present invention may beneficially be used for accelerating healing of bone fractures resulting from injuries or from surgical operations. According to specific embodiments the fractures are surgical fractures. In other embodiments, the compositions are orally administered for accelerating osteogenesis in osseointegration procedures.

The present invention is based in part on a surprising finding that oral administration of a pharmaceutical composition comprising stable amorphous calcium carbonate comprising at least one stabilizer accelerated healing of bone fractures during the distraction osteogenesis procedure. Specifically, it was found that the duration of the consolidation phase was shortened as compared to the accepted literature and to the data from the historical files of patients which underwent distraction osteogenesis without being administered calcium from the ACC source. The maturation rate of the new bone was evaluated using the bone healing index (BHI), which is defined as the time (days) needed for consolidation per cm of distracted osteotomy site (days/cm) and a decrease of 10% in BHI was found following administration of ACC during distraction osteogenesis procedure. Another ongoing clinical study is designed to evaluate the effect of the orally-administrable compositions comprising stable ACC for accelerating osteogenesis following distal-radius fractures. The orally-administrable compositions are contemplated to improve the functional outcome of the healing of the fractures, selected from fractures by external force, surgical fractures, pathological fractures or fatigue fractures.

Therefore, in one aspect, the invention provides a method of accelerating bone growth in a subject having a bone condition, selected from the group consisting of a fracture by external force, pathological fracture, fatigue fracture, distraction osteogenesis, osteotomy, osseointegration and combinations thereof, the method comprising orally administering to said subject an effective amount of a composition comprising stable amorphous calcium carbonate (ACC) comprising at least one stabilizer. Each possibility represents a separate embodiment of the invention. According to some embodiments, the method provides from about 0.25 to about 2 mm/day bone growth rate. According to other embodiments, the method provides from about 0.5 to about 1.5 mm/day bone growth rate. In certain embodiments, the method provides about 1 mm/day bone growth rate. According to further embodiments, the method provides enhancement of a functional outcome following the bone condition. According to still further embodiments, the method provides enhancement of the functional outcome following the fracture.

In certain embodiments, the bone condition comprises the distraction osteogenesis procedure. In further embodiments, the method comprises administering the composition comprising stable ACC comprising at least one stabilizer to the subject during the latency phase of the distraction osteogenesis procedure, the distraction phase of the distraction osteogenesis procedure, the consolidation phase of the distraction osteogenesis procedure or any combination thereof. Each possibility represents a separate embodiment of the invention. In some embodiments, the composition is administered during the latency phase of the distraction osteogenesis procedure. In other embodiments, the composition is administered during the distraction phase of the distraction osteogenesis procedure. In additional embodiments, the composition is administered during the consolidation phase of the distraction osteogenesis procedure. In further embodiments, the composition is administered during the distraction and the consolidation phases of the distraction osteogenesis procedure. In still further embodiments, the composition is administered during the latency, distraction and consolidation phases of the distraction osteogenesis procedure.

According to some embodiments, the composition is administered starting at least two days from the bone dissection of the distraction osteogenesis procedure. According to other embodiments, the composition is administered starting at least five days from the bone dissection of the distraction osteogenesis procedure. According to additional embodiments, the composition is administered starting at least ten days from the bone dissection of the distraction osteogenesis procedure.

In certain embodiments, the method comprises accelerating bone consolidation following bone distraction. In other embodiments, the method comprises accelerating bone consolidation following bone dissection.

In certain embodiments, the bone condition comprises the osseointegration procedure. In further embodiments, the method comprises administering the composition following the implant insertion stage of the osseointegration procedure, following the abutment insertion stage of the osseointegration procedure or a combination thereof. Each possibility represents a separate embodiment of the invention. In some embodiments, the osseointegration procedure comprises a dental implant integration. In further embodiments, the invention provides a method of accelerating bone growth in the osseointegration procedure of a dental implant in jawbone. In other embodiments, the osseointegration procedure comprises an orthopedic implant integration.

In some embodiments, the method comprises reducing the time of the implant's osseointegration. In other embodiments, the method comprises improving the quality of bone to implant contact.

In some embodiments, the invention provides a method of accelerating bone growth in a subject having a pathological fracture. According to further embodiments, the pathological fracture comprises a fracture associated with osteoporosis, osteomalacia, malignant tumor, multiple myeloma, osteogenesis imperfecta congenita, cystic bone, suppurative myelitis, osteopetrosis, nutrition disorders or a combination thereof. Each possibility represents a separate embodiment of the invention.

In some embodiments, the invention provides a method of accelerating bone growth in a subject having a fracture by external force. In other embodiments, the invention provides a method of accelerating bone repair in a subject having a fracture by external force.

According to further embodiments, the bone fractures comprise fractures of humerus, ulna, radius, femur, tibia, fibula, patella, ankle bones, wrist bones, carpals, metacarpals, phalanges, tarsals, metatarsals, ribs, sternum, vertebrae, scapula, clavicle, pelvis, sacrum and craniofacial bones. Each possibility represents a separate embodiment of the invention. In specific embodiments, distal radius fractures are treated with the compositions of the present invention. According to additional embodiments, the composition is administered to a subject having a non-union fracture, mal-union fracture or delayed union fracture. Each possibility represents a separate embodiment of the invention.

In some embodiments, the composition comprising stable ACC comprising at least one stabilizer is administered at least once a day. In further embodiments, the composition comprising stable ACC comprising at least one stabilizer is administered at least two times a day. In still further embodiments, the composition comprising stable ACC comprising at least one stabilizer is administered four times a day.

According to some embodiments, the method comprises administering said composition at a daily dose comprising from about 0.5 to about 5 g ACC. According to other embodiments, the method comprises administering said composition at a daily dose comprising from about 0.1 to about 1.5 g elemental calcium.

According to specific embodiments, the method of the present invention comprises administering of the compositions comprising stable ACC comprising at least one stabilizer in combination with vitamins. According to some embodiments, the vitamins are selected from the group consisting of Vitamin C, Vitamin D, Vitamin E and Vitamin K. Each possibility represents a separate embodiment of the invention. According to a specific embodiment, the composition comprising stable ACC comprising at least one stabilizer is administered in combination with Vitamin D₃. In some embodiments, the composition of the invention and the vitamins are administered simultaneously. In other embodiments, the composition of the invention and the vitamins are administered sequentially.

According to yet further embodiments, the subject in need of bone repair is selected from the group consisting of populations susceptible to the diminished bone fracture healing, including elderly, smokers, diabetics, osteoporotic patients; subjects suffering from protein malnutrition; and subjects consuming corticosteroids, chemotherapeutic agents, non-steroidal anti-inflammatory drugs, antibiotics, anticoagulants and medicants which reduce osteoclastic activity. Each possibility represents a separate embodiment of the invention. According to yet further embodiments, said subject is selected from patients that underwent osteotomy, osteoclasis, distraction osteogenesis or osseointegration surgeries.

In another aspect, the present invention provides an orally-administrable pharmaceutical composition comprising stable amorphous calcium carbonate (ACC) comprising at least one stabilizer, for use in accelerating bone growth in the treatment of a bone condition selected from the group consisting of a fracture by external source, pathological fracture, fatigue fracture, osteotomy, distraction osteogenesis, osseointegration and combinations thereof. Each possibility represents a separate embodiment of the invention. In some embodiments, said orally administrable composition is adapted to provide from about 0.5 to about 1.5 mm/day bone growth rate. In other embodiments, the orally-administrable pharmaceutical composition is for use in accelerating bone growth in the distraction osteogenesis procedure. According to further embodiments, the orally-administrable pharmaceutical composition comprising stable ACC comprising at least one stabilizer is for use in accelerating bone consolidation following bone distraction phase of the distraction osteogenesis procedure.

According to some embodiments, the composition of the present invention is for use during latency phase of the distraction osteogenesis procedure, the distraction phase of the distraction osteogenesis procedure, the consolidation phase of the distraction osteogenesis procedure or any combination thereof. In some embodiments, the composition is for use during the latency phase of the distraction osteogenesis procedure. In other embodiments, the composition is for use during the distraction phase of the distraction osteogenesis procedure. In additional embodiments, the composition is for use during the consolidation phase of the distraction osteogenesis procedure. In further embodiments, the composition is for use during the distraction and the consolidation phases of the distraction osteogenesis procedure. In still further embodiments, the composition is for use during the latency, distraction and consolidation phases of the distraction osteogenesis procedure.

In some embodiments, the orally-administrable pharmaceutical composition is for use in accelerating bone growth in the osseointegration procedure. The osseointegration procedure may comprise integration of a dental implant or an orthopedic implant. Each possibility represents a separate embodiment of the invention.

According to some embodiments the orally-administrable pharmaceutical composition comprises stable amorphous calcium carbonate comprising at least one stabilizer, wherein the stable ACC is of synthetic origin. According to other embodiments the stable ACC is of natural origin.

According to some embodiments the stabilizer is selected from the group consisting of organic acids, phosphoric or sulfuric esters of hydroxy carboxylic acids, hydroxyl bearing organic compounds, and combinations thereof. Each possibility represents a separate embodiment of the invention. In some embodiments, said stabilizer comprises at least one component selected from the group consisting of organic acids, phosphorylated organic acids, phosphoric or sulfuric esters of hydroxy carboxylic acids, phosphorylated amino acids and derivatives thereof, and hydroxyl bearing organic compounds. Each possibility represents a separate embodiment of the invention.

In some embodiments, said stabilizer comprises at least one component selected from phosphorylated amino acids. Said amino acids may be present in amino acid derivatives or oligopeptides or polypeptides. According to some embodiments the phosphorylated amino acids are selected from phosphoserine and phosphothreonine. In some embodiments, said stabilizer comprises at least one component selected from polyols. Said polyols may comprise alcohols or saccharides. Each possibility represents a separate embodiment of the invention. According to some embodiments, said stabilizer comprises at least one saccharide selected from mono-, di-, oligo-, and polysaccharides. According to some embodiments, said stabilizer comprises hydroxyl bearing organic compounds further combined with at least one alkali hydroxide. According to some embodiments, the stabilizer is a carboxylic acid. In further embodiments, the carboxylic acid is selected from the group consisting of citric acid, tartaric acid, malic acid and a combination thereof. In some embodiments, said stabilizer comprises at least one compound selected from phosphorylated amino acids, phosphorylated peptides, chitin with at least one peptide, and polyol with alkaline hydroxide.

According to some exemplary embodiments, said ACC is obtained from isolated crustacean gastroliths. According to one embodiment, said natural ACC is stabilized by chitin and polypeptides. According to other experimental embodiments, the ACC is synthetic, wherein said synthetic ACC is stabilized by phosphorylated amino acids selected from phosphoserine or phosphothreonine. According to another embodiment said synthetic ACC is stabilized by phosphoserine in combination with citric acid. According to still another embodiment, said synthetic ACC is stabilized by citric acid. According to yet another embodiment, said ACC is stabilized by sucrose in combination with sodium hydroxide.

The pharmaceutical composition according some embodiments may further comprise carriers, adjuvants, diluents, or excipients. The pharmaceutical composition may be formulated in a dosage form selected from the group consisting of tablets, capsules, pills, powders, granules, elixirs, tinctures, suspensions, syrups, emulsions and gels. In further embodiments, the orally-administrable pharmaceutical composition comprises a unit dose of about 0.2 to about lg ACC. According to additional embodiments, the orally-administrable pharmaceutical composition comprises a daily dose of about 0.5 to about 5 g ACC. In some embodiments, the orally-administrable pharmaceutical composition comprises a unit dose of about 0.05 to about 0.3 g of elemental calcium. According to further embodiments, the orally-administrable pharmaceutical composition comprises a daily dose of about 0.15 to about 1.5 g elemental calcium.

In yet another aspect, the present invention provides a use of stable amorphous calcium carbonate (ACC) comprising at least one stabilizer in accelerating bone growth in a subject having a bone condition, selected from the group consisting of a fracture by external force, pathological fracture, fatigue fracture, osteotomy, distraction osteogenesis, osseointegration and combinations thereof. Each possibility represents a separate embodiment of the invention.

In still another aspect, the present invention provides a use of stable amorphous calcium carbonate (ACC) comprising at least one stabilizer in the preparation of pharmaceutical compositions, nutraceutical compositions, or food supplements for accelerating bone growth in the treatment of bone conditions selected from the group consisting of a fracture by external force, pathological fracture, fatigue fracture, osteotomy, distraction osteogenesis, osseointegration and combinations thereof. Each possibility represents a separate embodiment of the invention.

All the above and other characteristics and advantages of the invention will be further understood through the following illustrative and non-limitative description of embodiments thereof, with reference to the appended drawings.

### BRIEF DESCRIPTION OF THE FIGURES

FIG. 1: Bone healing index (HI) of study and control groups.

Bars represent SEM.

FIG. 2: Cumulative percentage of healing index in study (•) and control (▪) groups.

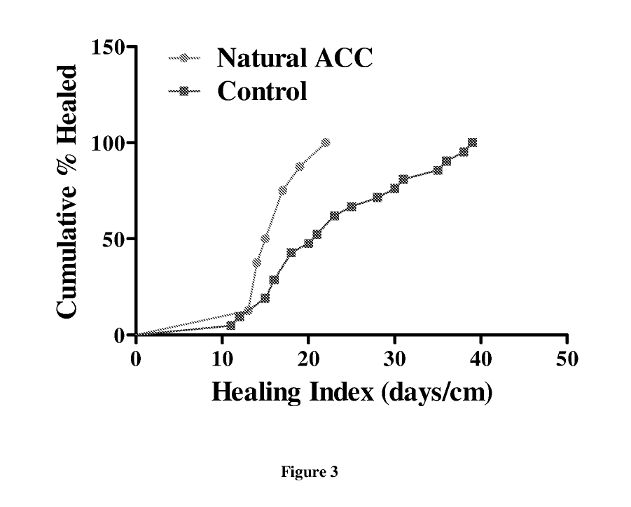

Log-rank test: p<0.007

FIG. 3: Bone healing index (BHI) of paired study and control subjects according to the lengthening performed values. Bars represent SEM.

### DETAILED DESCRIPTION OF THE INVENTION

The term “stable ACC” is used herein to indicate that the calcium carbonate is maintained in the amorphous state for long periods of time, e.g., from several weeks to several years, with no more than 5% conversion into the crystalline form over said period of time. The crystallization of the ACC is inhibited according to the invention by the addition of one or more stabilizer, selected from organic or inorganic ingredients such as phosphorylated amino acids, other organic acids, peptides, salts, saccharides, or lipids. Said salts may comprise, for example, cations selected from magnesium, potassium, strontium, and sodium, and anions selected from carbonate, phosphate, sulfate, chloride, bromide, fluoride, citrate, fumarate, malate or other organic anions; the terms anion and cation are used to simply describe the salt composition, without implying anything about the solubility or pH of the said molecules.

The terms “stabilizer” or “stabilizing agent” as used herein are used interchangeably and refer to any substance that preserves calcium carbonate in the amorphous state.

The term “effective amount” as used herein refers to a sufficient amount of the compositions comprising stable ACC to enhance bone fracture healing or bone implant integration at a reasonable benefit/risk ratio applicable to any medical or nutritional treatment.

The term “repair” as used herein refers to the bone fracture healing, including osteogenesis phase.

The term “bone condition” as used herein refers to any disease, condition, disorder, syndrome, or trauma of or to a bone which would be benefited, treated, rescued or healed by osteogenesis. Examples of such bone conditions include, but are not limited to, fracture, fracture by an external force, pathologic fractures, fatigue fractures, delayed unions, non-unions, malunions, distraction osteogenesis, osteotomy and osseointegration.

The term “fracture” as used herein refers to a fracture of a skeletal bone, whether simple or compound.

The term “distraction osteogenesis” as used herein refers to the process of lengthening bones or repairing skeletal deformities comprising increasing the size of a gap between sections of bone and allowing new bone to grow in said gap.

The term “osteotomy” as used herein refers to a surgical sectioning or surgical drilling of bone.

The term “osseointegration” as used herein refers to the firm anchoring of a surgical implant or prosthesis (as in dentistry or in bone surgery) by the growth of bone around or into said surgical implant or prosthesis without fibrous tissue formation at the interface of said bone and said surgical implant or prosthesis.

The terms “osteogenesis” or “bone growth” that can be used interchangeably, as used in some embodiments, refer to an increase in the presence and activity of osteoblasts and the direct formation of bone tissue by said osteoblasts.

The amorphous calcium carbonate based compositions according to the present invention were found to be superior calcium sources over commonly marketed calcium supplements during the bone repair process. Compositions comprising ACC have a surprising effect on active mineralization during bone remodeling thus accelerating fracture healing process. According to some embodiments, the orally-administrable compositions of the present invention are adapted to provide bone growth rate of from about 0.5 to about 1.5 mm per day, preferably from about 0.75 to about 1.25 mm per day, more preferably about 1 mm per day. The compositions of the present invention are useful in bone repair following fractures by external force, surgical fractures, pathological fractures, and fatigue fractures. The orally-administrable compositions are also useful in stimulation of osteoclasis in osseointegration procedures, such as dental or orthopedic prostheses implantation.

Therefore, in some embodiments, the present invention provides a pharmaceutical composition comprising stable amorphous calcium carbonate (ACC) comprising at least one stabilizer selected from the group consisting of organic acids, phosphorylated organic acids, phosphoric or sulfuric esters of hydroxy carboxylic acids, phosphorylated amino acids and derivatives thereof, and hydroxyl bearing organic compounds, for use in accelerating repair of bone fractures. In other embodiments, the invention provides a method of accelerating bone repair in a subject in need, comprising administering to said subject an effective amount of a composition comprising stable amorphous calcium carbonate (ACC) comprising at least one stabilizer. In further embodiments, the invention provides a method of accelerating bone growth in a subject in need, comprising administering to said subject an effective amount of said composition. According to some embodiments, the bone repair is subsequent to a fracture, selected from fracture by external force, surgical fracture, pathological fracture, and fatigue fracture. According to specific embodiments, the subject in need suffers from a bone fracture, caused by surgical fracture. According to some embodiments, the surgical fracture comprises fractures originating from osteotomy, osteoclasis, and distraction osteogenesis surgeries. According to further embodiments, the bone repair in a subject suffering from bone fracture further comprises enhancing functional outcome following fracture.

According to additional embodiments, the invention provides a method of accelerating bone growth in a subject having a bone fracture selected from the group consisting of a fracture by external force, surgical fracture, pathological fracture, fatigue fracture and a combination thereof, the method comprising orally administering to said subject an effective amount of a composition comprising stable amorphous calcium carbonate (ACC) comprising at least one stabilizer. According to other embodiments, the invention provides a method of accelerating bone growth in osseointegration, the method comprising orally administering to a subject undergoing osseointegration procedure an effective amount of a composition comprising stable amorphous calcium carbonate (ACC) comprising at least one stabilizer.

According to further embodiments, the present invention provides compositions comprising stable amorphous calcium carbonate, comprising at least one stabilizer for use in accelerating bone growth in a subject having a bone condition selected from the group consisting of a fracture by external force, pathological fracture, fatigue fracture, osteotomy, distraction osteogenesis, osseointegration and combinations thereof. In another aspect, the present invention provides a method of accelerating bone growth in a subject having said bone condition, the method comprising administering to said subject an effective amount of the composition comprising stable amorphous calcium carbonate, comprising at least one stabilizer.

Methods of Treatment

The use of the compositions comprising stable ACC is particularly beneficial for the acceleration of bone growth following surgical bone fractures. The compositions of the present invention may be used for treating surgical fractures selected from, but not limited to the fractures originating from osteotomy, osteoclasis, and distraction osteogenesis procedures.

In some embodiments, the compositions of the present invention are used to accelerate bone growth of a fracture following osteotomy or osteoclasis surgery. Osteotomy and osteoclasis are surgical operations wherein a bone is cut to shorten, lengthen, or change its alignment. Said operations may be performed to correct a deformity of a bone. The non-limiting examples of the osteotomy surgeries are corrections of coxa vara, genu valgum, and genu varum, and osteotomies of the hip, knee, jaw, and chin. Osteotomy may also be performed to straighten a bone that has healed crookedly following a fracture.

In other embodiments, the compositions of the present invention are used to accelerate bone growth of a fracture following distraction osteogenesis surgery. The distraction osteogenesis procedure is a surgical process used to reconstruct skeletal deformities and lengthen the long bones of the body. A corticotomy is used to fracture the bone into two segments, and the two bone ends of the bone are gradually moved apart during the distraction phase, allowing new bone to form in the gap. When the desired or possible length is reached, a consolidation phase follows in which the bone is allowed to keep healing. Distraction osteogenesis has the benefit of simultaneously increasing bone length and the volume of surrounding soft tissues. The distraction osteogenesis procedure can be used in the field of orthopedics and/or dentofacial orthopedics, for example in lower and upper limb lengthening or in the correction of micrognathia, midface, and fronto-orbital hypoplasia in patients with craniofacial deformities. The Ilizarov method, the oldest and most common method of the limb lengthening by distraction osteogenesis consists of the following stages:
1. Percutaneous cortex dissection—the bone is broken into two segments by a subperiosteal osteotomy aiming at the maximal preservation of the intramedullar and periosteal blood supply as far as possible.
2. Stabilization of the elongating bone by an external fixator with an orbicular frame-distracter (Ilizarov apparatus), which pierces through skin and muscles into the bone. The distracter is fitted in a way that guarantees the ideal biomechanical circumstances for an even osteogenesis, such as complete immobility at the osteotomy site, and the potential of force application.
3. Latency phase—callus distraction is better promoted by a waiting period of five to ten days after the osteotomy. Waiting period longer than fifteen to twenty days increases the risk of early callus maturation. The waiting period till distraction begins, gives sufficient time for the rehabilitation of the soft tissue damage, and facilitates, the best possible way, the biology of callus distraction.
4. Distraction period—Screws attached to the middle bone are turned 1 millimeter (mm) per day, so that new bone tissues that are formed in the growth zone are gradually pulled apart to increase the gap. Lengthening of one millimeter per day is ideal for the osteogenesis procedure. Distraction rate of four times per day (0.25 mm every six hours) is markedly beneficial. The distraction speed and rate are modified according to the age of the patient, the bone quality and the degree of soft tissue injury.
5. Consolidation phase—The new bone stabilizes after the lengthening. Consolidation phase often lasts twice as long as the distraction phase in children and three to four times as long as the distraction phase in adults. During this phase the bone is no longer distracted but the patient continues to wear the external distracter.
6. Full limb loading in order to promote bone maturation. During this period the patient gradually regains use of the operated limb.

The compositions of the invention may be administered to a subject during the latency phase, the distraction phase, the consolidation phase or any combination thereof. Each possibility represents a separate embodiment of the invention. In some exemplary embodiments, the method of accelerating the bone growth during the distraction osteogenesis procedure included administration of the orally-administrable composition comprising stable ACC comprising at least one stabilizer during distraction and consolidation phases. In other experimental embodiments, the method comprised administration of the composition during the latency, distraction and consolidation phases.

The administration of the compositions may be started before the distraction osteogenesis surgery. Alternatively, the administration of the compositions may be started on the same day of the distraction osteogenesis surgery. In other embodiments, the administration is started at least one day following the surgery. In alternative embodiments, the compositions are administered starting two, five, ten, fifteen or twenty days from the distraction osteogenesis surgery. Each possibility represents a separate embodiment of the invention.

The compositions of the present invention can be used for accelerating bone consolidation following bone distraction. In these embodiments, the compositions can be administered before the beginning of the consolidation phase or before the beginning of the distraction phase. The compositions of the present invention are further adapted to reduce the consolidation phase following the bone distraction.

The compositions of the present invention can further be used for accelerating bone growth during an osseointegration procedure.

The osseointegration surgery generally comprises two surgical interventions and two healing phases following the surgical interventions. During the initial stage, a specially designed implant is threaded into the medullary cavity and the wound is completely closed. After Stage 1 surgery, which is a healing phase of osseointegration, it is important that the implant not be loaded until the bone has grown into the threads. The waiting time after the Stage 1 surgery, during which the osseointegration takes place, is normally six months, although in dental applications the loading of the implant may start earlier. During Stage 2 surgery, the implanted fixture is reexposed and an abutment is connected to the fixture. Stage 2 surgery is followed by a mobilization phase, which typically takes additional six months. During this phase the prosthesis is gradually loaded until the implant can accept full body weight.

In some embodiments of the invention, the orally-administrable composition is administered following the implant insertion stage. In other embodiments, the orally-administrable composition is administered following the abutment insertion stage. The orally administrable composition can be administered following both stages.

The compositions of the present invention may be used for bone growth acceleration in dental implants osseointegration. Additionally or alternatively, the compositions may be used for bone growth acceleration in orthopedic implants osseointegration. The implants may include a variety of biocompatible structures designed to engage the skeletal structure of the body to replace or support a bone structure, including specifically dental implants, craniofacial structures and bone and joint replacement component parts.

The compositions of the present invention may further be used in accelerating bone growth in fractures selected from, but not limited, to the fractures of humerus, ulna, radius, femur, tibia, fibula, patella, ankle bones, wrist bones, carpals, metacarpals, phalanges, tarsals, metatarsals, ribs, sternum, vertebrae, scapula, clavicle, pelvis, sacrum and craniofacial bones. In specific embodiments, distal radius fractures are treated with the compositions of the present invention. Compositions of the present invention may be administered to a subject suffering from any of the following fractures, including fissure fracture, greenstick fracture, transverse fracture, oblique fracture, spiral fracture, segmental fracture, comminuted fracture, avulsion fracture, compression fracture, depression fracture, and the like. Administering of the stable ACC is particularly beneficial in cases of non-union fractures, mal-union fractures, or delayed union fractures. Said fractured may be an external force fractures or pathological fractures.

The pathological fractures may further be selected from, but not limited to, fractures associated with osteoporosis, osteomalacia, malignant tumor, multiple myeloma, osteogenesis imperfecta congenita, cystic bone, suppurative myelitis, osteopetrosis, and nutrition disorders.

The method and the composition of the present invention further provide enhancement of a functional outcome following the bone fracture healing. For example, following the bone fracture of a limb, significant deficits in strength and motion of the limb may appear despite apparently successful repair of the fracture. The present invention provides a method for the enhancement of the functional outcome following bone fractures healing, comprising administering to a subject having a bone fracture an effective amount of a composition comprising stable amorphous calcium carbonate, comprising at least one stabilizer.

The methods of the present invention include administering of the compositions comprising stable amorphous calcium carbonate, comprising at least one stabilizer on a daily basis. In some embodiments, the compositions are administered at least one time a day. In other embodiments, the compositions are administered 1-4 times a day.

The compositions of the present invention may be administered in combination with other medications for treatment of bone fractures. According to some embodiments, said compositions are administered in combination with other minerals. The minerals are selected from Zinc, Copper, Calcium, Phosphorus, and Silicon. The compositions comprising ACC may further be administered in combination with vitamins. The vitamins are selected from, but not limited to, Vitamin C, Vitamin D, Vitamin E or Vitamin K Vitamin D may comprise Vitamin D₁, Vitamin D₂, Vitamin D₃, Vitamin D₄, Vitamin D₅ or a combination thereof. According to one embodiment, the composition comprising stable ACC comprising at least one stabilizer is administered in combination with Vitamin D₃. In the context of the present invention the term “combination therapy” encompasses administration of two or more active ingredients in a single dosage form or in separate dosage forms. Separate dosage forms may be administered simultaneously or sequentially or on entirely independent separate regimens. For example, the ACC may be administered daily and Vitamin D₃ may be administered less frequently.

According to some embodiments, the compositions of the present invention are administered to populations susceptible to the diminished bone fracture healing, including elderly, smokers, diabetics, osteoporotic patients, subjects suffering from protein malnutrition, and subjects consuming corticosteroids, chemotherapeutic agents, non-steroidal anti-inflammatory drugs, antibiotics, anticoagulants and medicants which reduce osteoclastic activity. According to other embodiments, the subjects in need of accelerated bone repair are selected from patients after osteotomy, osteoclasis, and distraction osteogenesis surgeries.

The Compositions Comprising Stable ACC

In some embodiments, the present invention provides a synthetic (artificial) composition comprising stable ACC and an amount of a stabilizer sufficient to maintain the ACC in a non-crystalline state. The stabilizer is selected from, but not limited to, organic acids, phosphorylated organic acids, phosphoric or sulfuric esters of hydroxy carboxylic acids, phosphorylated amino acids and derivatives thereof, and hydroxyl bearing organic compounds. According to some embodiments, said stabilizer comprises at least one component selected from phosphoric or sulfuric esters of hydroxyl carboxylic acids, such as phosphoenolpyruvate, phosphoserine, phosphothreonine, sulfoserine or sulfothreonine and hydroxyl bearing organic compounds, selected from mono-, di-, tri-, oligo- and poly-saccharides, for example, sucrose, mannose, glucose. The stabilizer comprising hydroxyl bearing compound may further comprise at least one alkali hydroxide, such as sodium hydroxide, potassium hydroxide and the like. In some embodiments of the invention, said stabilizer is selected from phosphorylated amino acids and polyols. The phosphorylated acids may be present in oligopeptides and polypeptides. In other embodiments of the invention, the stabilizer is an organic acid, preferably a carboxylic acid. The carboxylic acid is preferably selected from citric acid, tartaric acid or malic acid. According to an experimental embodiment, the synthetic composition comprises stable ACC, comprising citric acid.

In another embodiment, the present invention provides a natural composition, comprising stable ACC obtained from isolated crayfish gastrolith. The ground gastrolith comprises ACC, organic matter consisting mainly of chitin and polypeptides, and salts. The components present in the organic matter stabilize the ACC and prevent its crystallization.

A particular advantage of the compositions according to the invention is their confirmed low toxicity and high safety for oral administration. The compositions of the invention may be preferably administered orally in various oral forms including, but not limited to, tablets, capsules, pills, powders, granules, elixirs, tinctures, suspensions, syrups, emulsions and as gel form. In instances in which oral administration is in the form of a tablet or capsule, the composition components can be combined with a non-toxic pharmaceutically acceptable inert carrier or excipients such as lactose, starch, sucrose, glucose, modified sugars, modified starches, methylcellulose and its derivatives, mannitol, sorbitol, and other reducing and non-reducing sugars, magnesium stearate, stearic acid, sodium stearyl fumarate, glyceryl behenate, amorphous silica gel or other desiccant material and the like.

The compositions of the invention may typically be administered in daily doses of from about 0.5 to about 5 g ACC. According to alternative embodiments, the compositions of the invention may typically be administered in daily doses of from about 2 to about 3 g ACC. The compositions of the invention may typically be administered in unit doses of from about 0.2 to about 1 g ACC. The compositions of the invention may typically be administered in daily doses comprising elemental calcium in a range of from about 0.15 to about 1.5 g. According to alternative embodiments, the compositions of the invention may typically be administered in daily doses comprising elemental calcium in a range of from about 0.5 to about 1 g. In some embodiments, the compositions of the invention are administered in unit doses comprising from about 0.05 to about 0.3 g elemental calcium. Elemental calcium from ACC may be administered in lower doses compared to the crystalline calcium carbonate due to its higher efficacy in bone mineralization process and therefore a larger contribution to the fracture healing process.

For oral administration in liquid form, the composition components can be combined with non-toxic pharmaceutically acceptable inert carriers such as ethanol, glycerol, water and the like. When desired or required, suitable binders, lubricants, disintegrating agents and coloring and flavoring agents can also be incorporated into the mixture.

The inventors have conducted experiments aiming to evaluate the efficacy of compositions comprising stable ACC in repairing bone surgical fractures. The inventors have further conducted experiments aiming to evaluate the efficacy of compositions comprising stable

ACC in repairing unintentional bone fractures. The bone repair is assessed both in terms of the time needed for a fracture to heal completely and by evaluating the functional outcome following the fracture. The bone fracture healing is evaluated by radiographic examination by three or four independent reviewers, as well as image analysis tools. The functional outcome following the fracture is evaluated by means of completion of both the limb-specific questionnaire (Disabilities of the Arm, Shoulder and Hand (DASH)) and the Visual Analogue Scale (VAS) questionnaire and by assessment of the pain-free grip and force plate tests.

### EXAMPLES

### Example 1

### Natural ACC Effect on Bone Repair During Distraction Osteogenesis

The study was a prospective, unblinded trial to test the safety and efficacy of administering stable amorphous calcium carbonate obtained from isolated gastrolith during distraction osteogenesis performed by the Ilizarov method as described hereinabove. The major follow-up method for the distraction osteogenesis is simple X-rays. X-rays were performed during the 10th-15th postoperative day in order to confirm the presence of early callus at the osteotomy site and to start the distraction.

The trial included a study group of 10 patients (ages 4-15) who underwent distraction osteogenesis surgery. Recovery of the operated bone was assessed by radiographs. During the last week of the distraction phase and the entire consolidation phase until the removal of the external fixator, the patients in the study group consumed ACC powder, obtained from isolated gastrolith, daily. The maturation rate of the new bone was evaluated using the bone healing index (BHI), which is defined as the time (days) needed for consolidation per cm of distracted osteotomy site (days/cm). The data collected from the study group administered with ACC powder was compared with data from historical files of 21 untreated patients (ages 5-16) that underwent distraction osteogenesis.

Experimental Details

Investigational Product

Table 1 summarizes the chemical analysis of ACC powder obtained from isolated gastrolith using Inductively Coupled Plasma Atomic Emission (ICP-AE), Ultraviolet (UV) Spectroscopy, Loss on Ignition (LOI) and flame photometer. The analysis was performed by two independent laboratories: Ben-Gurion University, Beer-Sheva, Israel and Ernie Miller labs Ltd., Beer-Sheva, Israel. Unless otherwise specified, the accuracy of the measured values is ±10%.

On the last week of the distraction phase and every month thereafter, patients were provided with a monthly supply of the ACC powder in a sealed container—65 capsules of 500 mg each for oral use. Each capsule comprised 125±5 g elemental calcium. Starting on the first day of the last week of the distraction phase, during the entire consolidation phase until the external fixator is removed; all subjects consumed two (2) 500 mg capsules a day (total of 1 g of the ACC powder and about 250 mg of elemental calcium) along with lg sour sugar (for compliance). The container was kept in the refrigerator (˜4° C.).

TABLE 1
Chemical analysis of Amorphical ACC
obtained from isolated gastrolith.

| Ernie Miller labs. | Ben Gurion | |
|---|---|---|
| Ltd. | University | |
| Composition [%] | Composition [%] | The Element |
| 57.6 | 65 | Calcium carbonate (CaCO₃) |
| *Calcium soluble in | Calcium (Ca⁺²)- 26 | |
| acid (CaO) - 32.3 | Carbonate(CO₃⁻²) | |
| **Carbonate (as CO₂) | | |
| 7 | ND | Carbonate (not bound to |
| | | Ca⁺²) |
| 13 | Within the organic | Moisture (crystal water) |
| 11.1 | 26 | Organic (biologic) matter |
| (2.2) | 1.3 | Phosphorus (P⁻³) |
| (as MgO⁻) 0.7 | 0.53 | Magnesium (Mg⁺²) |
| 2.3 | 0.45 | Sodium (Na⁺¹) |
| 0.01 | 0.39 | Chlorine (Cl⁻¹) |
| <0.01 | 0.14 | Sulfur (S⁻²) |
| N.D. | 0.11 | Fluorine (F⁻¹) |
| 1.9 | 0.03 | Potassium (K⁺¹) |
| 0.28 | N.D. | Strontium (Sr⁺²) |
| 99.2 | 93.95 | Total |

Study Design

Each patient (and his parent in case of children) met the eligibility criteria and provided signed informed consent prior to entering the study. Patients underwent the standard surgical procedure and were administrated with the supplement in the last week of the consolidation phase and till the removal of the external fixator.

The follow up was performed in the same manner as the routine follow up which is customary in the department. The patients and the physicians were asked to fill questionnaires.

The duration of the consolidation phase, adjusted to the extent of lengthening performed, was measured to test if the ACC powder supplementation facilitated bone mineralization and shortened the consolidation phase compared to the accepted literature and to the data from the historical files of patients which underwent distraction osteogenesis without being administered calcium from the ACC source.

All clinical decisions were made by the treating orthopedic surgeon disregarding participation in the study. Analyses of the data were based on the clinical data and radiographs obtained during distraction and consolidation process.

All the patients, with the help of the research staff, filled out a questionnaire regarding their general health before the surgery, during hospitalization and after removal of the external fixator. This data was recorded and combined with the surgeon's data concerning clinical course, complications including pin site and other infections, pin breakage, etc.

Radiographic Evaluation

Radiographs were taken on a weekly basis routinely during the lengthening phase and biweekly during consolidation phase or as necessary clinically. To allow for consistency, X-ray exposure parameters were determined for each patient at the beginning of the process and were used in each radiograph thereafter. The radiographs were analyzed by three additional orthopedic surgeons familiar with limb lengthening and blinded to the timing of the radiographs. These surgeons reviewed all patients' radiographs and determined independently the optimal time for external fixator removal. The duration of the fracture healing was compared to the healing index of the historical reference of the medical center.

Statistical Analysis

Results are expressed herein as means±SEM, One-way ANOVA and Fisher-LSD post hoc comparison tests were performed using STATISTICA 6.1 software (StaSoft, Tulsa, Okla.). A p value<0.05 was deemed significant.

Results

Table 2 represents data collected from the study group administered with the ACC powder and data from historical files of 21 untreated patients.

The primary objective of the trial was to test the effect of the ACC powder nutraceutical on mineralization rate during distraction osteogenesis, with the hypothesis that daily oral administration of ACC powder will facilitate bone mineralization during the consolidation phase of distraction osteogenesis and shorten treatment time, thereby providing a method of accelerating the repair of bone fractures, particularly surgical fractures.

TABLE 2
Data collected from patients who underwent distraction osteogenesis procedure.

| | Age at | | Operated | | Operation | Total # of | Total # of | Lengthening | Bone Healing |
|---|---|---|---|---|---|---|---|---|---|
| Group | surgery | Gender | leg | femur/tibia | date | lengthening days | healing days | extent (cm) | Index |
| Study | 7 | M | R + L | T | 03/11/2010 | 99 | 77 | 4 | 19 |
| | 15 | F | R + L | F | 24/11/2010 | 85 | 91 | 7 | 13 |
| | 14 | M | L | F | 16/03/2011 | 75 | 101 | 6 | 17 |
| | 14 | M | L | F | 23/03/2011 | 71 | 98 | 7 | 14 |
| | 9 | F | R + L | F | 13/04/2011 | 52 | 71 | 5 | 14 |
| | 14 | M | R + L | T | 15/06/2011 | 47 | 133 | 6 | 22 |
| | 8 | F | R + L | T | 06/07/2011 | 40 | 84 | 5 | 17 |
| | 9 | M | R + L | F | 17/08/2011 | 61 | 105 | 7 | 15 |
| | 4 | M | L | F | 04/01/2012 | 48 | 77 | 4.5 | 17 |
| Control | 5 | F | R | T | 14/12/2005 | 103 | 98 | 4 | 25 |
| | 14 | M | R + L | T | 23/06/2010 | 54 | 98 | 5.5 | 18 |
| | 14 | F | L | T | 25/06/2008 | 47 | 105 | 3 | 35 |
| | 13 | M | L | F | 27/05/2009 | 89 | 70 | 3 | 23 |
| | 6 | M | L | F | 24/02/2010 | 61 | 59 | 4 | 15 |
| | 9 | F | R | F | 23/07/2008 | 85 | 109 | 6 | 18 |
| | 14 | F | L | F | 30/12/2009 | 78 | 77 | 6.5 | 12 |
| | 9 | M | L | T | 09/07/2003 | 25 | 115 | 3 | 38 |
| | 13 | F | R | F | 12/07/2006 | 78 | 55 | 3 | 18 |
| | 12 | F | R | F | 06/07/2005 | 114 | 141 | 7 | 20 |
| | 12 | M | L | T | 14/05/2008 | 109 | 113 | 4 | 28 |
| | 14 | M | L | F | 05/07/2006 | 63 | 54 | 3.5 | 15 |
| | 5 | M | R | T | 26/11/2008 | 68 | 57 | 5 | 11 |
| | 11 | F | R + L | T | 23/07/2008 | 41 | 121 | 3.4 | 36 |
| | 11 | F | R + L | T | 23/07/2008 | 41 | 121 | 3.1 | 39 |
| | 16 | M | L | F | 28/06/2006 | 104 | 108 | 3.5 | 31 |
| | 15 | F | L | F | 13/06/2007 | 96 | 127 | 5 | 25 |
| | 16 | M | R | F | 07/02/2010 | 42 | 85 | 4 | 21 |
| | 6 | F | R + L | F | 23/12/2009 | 82 | 94 | 6 | 16 |
| | 16 | M | R | F | 28/08/2002 | 68 | 70 | 4.5 | 16 |
| | 16 | F | R | F | 01/05/2002 | 61 | 91 | 3 | 30 |

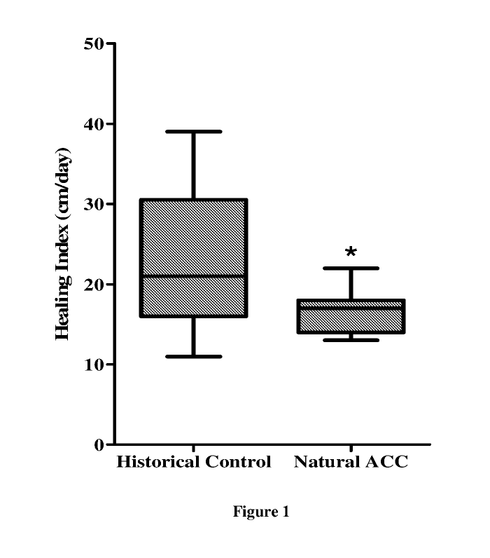

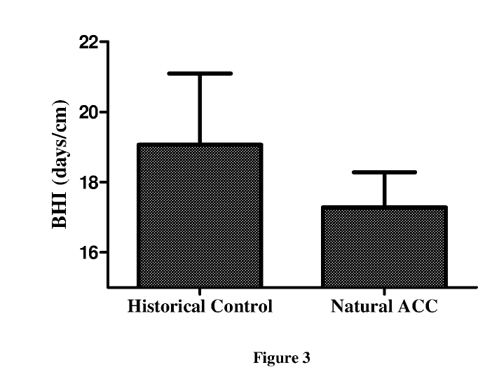

Mean bone healing index (BHI) in the natural ACC (gastrolith) treated group was 16 days/cm (range 13-22), compared to 22 days/cm (range 11-38). The difference was statistically significant (P<0.02; FIGS. 1 & 2). Also, the mean standard deviation for the study group was significantly lower compared to the control group (2.88 and 8.15, respectively), implying for standardization of the healing process. Due to the fact that BHI is known to be affected by the extent of lengthening performed, 7 subjects with similar extent of lengthening from each group were compared (FIG. 3). Paired comparison showed an overall 10% decrease in BHI following natural ACC (gastrolith) treatment, independent of the performed lengthening value.

The results of the above trial confirmed that administering stable ACC derived from isolated crustacean gastrolith accelerates bone fracture healing and provides a more standardized healing process.

### Example 2

### Synthetic ACC Effect on Bone Repair During Distraction Osteogenesis

The experiment aiming to evaluate the efficacy of administering synthetic stable amorphous calcium carbonate, comprising at least one stabilizer, during distraction osteogenesis is conducted. A multicenter prospective, randomized, parallel, double-blind, active controlled study compares the effect of amorphous calcium carbonate (ACC) versus crystalline calcium carbonate (CCC) on bone healing time following leg bone dissection and lengthening by distraction osteogenesis.

The primary objective of the trial is to assess the efficacy of treatment with calcium from ACC compared to CCC on bone healing index in subjects that underwent leg lengthening by distraction osteogenesis. The secondary objective is to evaluate the effect of ACC compared to CCC on the improvement in functional outcome following distraction osteogenesis. The further secondary objective is to evaluate the safety profile of ACC in this population.

Selection of Study Population

The study population includes 40 subjects that underwent distraction osteogenesis lengthening, twenty (20) subjects in each treatment group.

Inclusion Criteria:
Subjects that intend to undergo limb lengthening by distraction osteogenesis procedure.
External fixator.
Age 4-30 (inclusive)
Subjects able to adhere to the visit schedule and protocol requirements and be available to complete the study.
Subjects that had signed the ICF.

Exclusion Criteria:
Elevated serum calcium (>10.2 mg/dL)
Subjects suffering from active liver disease or clinical jaundice
Subjects with current or a history of a malignant neoplasm in the 5 years prior to the study
Cognitive impairment
Any disease affecting thyroid or parathyroid glands
Chronic renal failure
Any known diseases affecting the absorption from the gastrointestinal tract
Active metabolic bone disease
Glucocorticoid therapy.
Pregnant or breast feeding women

Description of the Investigational Product

TABLE 3
Chemical analysis of Amorphical synthetic stable ACC.

| Analysis | USP requirements | ACC analysis result |
|---|---|---|
| Loss On Ignition | N/A | 14.8% |
| (LOI) | | |
| Ethanol residues | N/A | 0.034% |
| Acid Insoluble | Less than 0.2% | Less than 0.0002% |
| Calcium | N/A | 32.5% |
| Chlorides | N/A | 1.36% |
| Sodium | N/A | 1.85% |
| Phosphorus | N/A | 0.143% |
| Sulfur | N/A | <0.1% |
| Iron | Less than 1,000 ppm | 9.5 ppm |
| Alkali metals* | Less than 10,000 ppm | 475 ppm |
| Barium** | Pass flame test | 20 ppm |
| Mercury | Less than 0.5 ppm | Less than 1 ppm*** |
| Fluorides | Less than 0.005% | 0.001% |
| Lead | Less than 3 ppm | Less than 5 ppm*** |
| Heavy metals | Less than 0.002% | Trace (Less than 0.002%) |
| Arsenic (total) | Less than 3 ppm | Less than 3 ppm |
| Crystalline | Less than 5% | Less than 1% |
| Calcium | | |
| Carbonate | | |

*Not including sodium.
**Cannot be performed according to the USP (was performed in Inductive Coupled Plasma (ICP)).
***Interferences in the ICP measurements prevented lower concentration analyses.

The stable amorphous calcium carbonate used in the study is a synthetic ACC stabilized by low concentrations of phosphoserine and citrate (less than 0.5% in the final product), provided by Amorphical Ltd. Phosphorylated serine and the organic citric acid are non-toxic, naturally abundant and even consumed as a standalone dietary supplements with no reported adverse effects when taken orally.

Table 3 summarizes the ACC chemical analysis, assessed according to the U.S. Pharmacopeia parameters of calcium carbonate, using Inductively Coupled Plasma Atomic Emission (ICP-AE), Ultraviolet (UV) Spectroscopy, Loss on Ignition (LOI) and flame photometer.

Description of the Control Product

The control product contained in each tablet 500 mg of crystalline calcium carbonate (200±5 mg elemental calcium) and 167 mg of sucrose. Elemental calcium level in the control product was equal to the elemental calcium level in the treatment group in order to evaluate the effect of amorphous calcium carbonate compared to crystalline calcium carbonate.

Dosage and Administration

Eligible subjects have randomly received one of the two treatments. Each dose of the study supplement consisted of 640 mg of ACC, comprising 200 mg elemental calcium or 500 mg CCC in each tablet, as presented in table 4.

TABLE 4
Dose of Study Treatment

| Group | Treatment |
|---|---|
| Investigational | Tablets for oral use containing 640 mg ACC (200 |
| Product | mg elemental calcium) |
| Control Product | Tablets for oral use containing 500 mg CCC (200 |
| | mg elemental calcium) and 167 mg of sucrose. |

Selection of the dosage is based on the recommended daily intake of elemental calcium for subjects of age 19-50 (1000 mg/day). The calcium dosage in the control is equaled to the elemental calcium levels in ACC product.

Allocation of Subjects to Treatment

Subjects are assigned to one of the treatment groups randomly according to a randomization list. Randomization to each of the two study arms is performed using block randomization within center.

Blinding

The oral calcium treatments administered in the clinical trial are blinded. The subjects, the investigators and any personnel involved in subjects' assessment, monitoring, analysis and data management are blinded to the subject formulation assignment, except the Sponsor who is responsible for preparing, dispensing and labeling the investigational product. Blinded labels are affixed to the vials prior to dosing by the un-blinded Sponsor.

Randomization Procedures

The study is double blinded and therefore the CRC staff and the subject remain blinded to the code assignments throughout the study. Prior to administration, each subject is assigned with an individual number and is treated according to the predetermined computer generated randomization list. A computer-generated algorithm is used to assign the subject into the treatment groups. The treatment compositions are prepared by the Sponsor and labels are affixed to the vials prior to shipping. The hospital pharmacists are instructed to dispense the products to the CRC according to the cohort assignment lists.

Study Design

Forty (40) subjects are randomly assigned to one of two groups (N=20). Subjects in the treatment group receive amorphous calcium carbonate (ACC) and those in the active control group receive crystalline calcium carbonate (CCC). Both formulations are supplemented with vitamin D

Safety parameters are evaluated throughout the trial.

Subjects admitting to the medical center for distraction osteogenesis surgery are routinely evaluated. Eligible subjects, complying with all inclusion criteria and none of the exclusion criteria, are considered candidates for the trial and are invited to the CRC for screening.

Screening (Day −7)—Subjects (and subjects' parents in case of children) sign an informed consent form (ICF). Chemistry and hematology tests are performed: sodium, potassium, hemoglobin, sedimentation rate, leukocytes calcium (total, albumin-corrected), phosphate, alkaline phosphatase, creatinine, and albumin. Also, serum PTH, 25-hydroxyvitamin D, and thyroid-stimulating hormone (TSH) are tested. Urinary excretion of calcium and creatinine is measured. General health is examined by medical history and physical examinations. Eligible subjects, complying with all inclusion criteria and none of the exclusion criteria are enrolled to the study. Subjects are informed by phone or on site whether they are eligible to enter the study.

Pre-Surgery

Visit 1 (Day 0)—Eligible subjects are invited to the CRC prior to the surgery. X-ray is performed at baseline.

Distraction Phase

Visit 1 (Day 7)—Controlled antero posterior and lateral x-ray is performed. Subjects randomly receive packs of tablets, each tablet containing 200 mg elemental calcium (a total of 100 tablets, 21 day supply+16 spare tablets) with one of the formulations (ACC or CCC). Subjects are instructed to begin the treatment 2 days from the surgery. Subjects are instructed to take 4 tablets a day. Subjects are advised to take vitamin D3 supplementation based on the doctors' decision.

Visit 2—(Day 14±1)—Controlled antero posterior and lateral x-ray is performed. Subjects are asked about any side effects or AEs that may have occurred.

Visit 3—(Day 21±1)—Controlled antero posterior and lateral x-ray is performed. Subjects are advised to take vitamin D3 supplementation based on the doctors' decision.

Visit 4—(Day 28±1)—Controlled antero posterior and lateral x-ray are performed. Subjects are asked about any side effects or AEs that may have occurred. Subjects receive additional packs of tablets, each containing 200 mg elemental calcium (a total of 100 tablets, 21 day supply+16 spare tablets) with the same formulation received on day 0. Subjects are instructed to take 4 tablets a day. Subjects that did not complete the distraction phase are invited for an additional radiographic assessment based on the doctors' decision.

Consolidation Phase

Visit 1 (Day 0)—Controlled antero posterior and lateral x-ray is taken. Functional assessment measurements are performed. Subjects are asked about any side effects or AEs that may have occurred. Subjects receive additional packs of tablets, each containing 200 mg elemental calcium (a total of 100 tablets, 21 day supply+16 spare tablets) with the same formulation received in day 0. Subjects are instructed to take 4 tablets a day. Subjects that did not complete the distraction phase are invited for an additional radiographic assessment based on the doctors' decision.

Visit 2 (Day 14±1)—Controlled antero posterior and lateral x-ray is taken. Functional assessment measurements are performed. Subjects receive additional packs of tablets, each containing 200 mg elemental calcium (a total of 100 tablets, 21 day supply+16 spare tablets) with the same formulation received in day 0. Subjects are instructed to take 4 tablets a day. Subjects that did not complete the distraction phase are invited for an additional radiographic assessment based on the doctors' decision.

Visit 3—(Day 35±1)—Controlled antero posterior and lateral x-ray is performed. Functional assessment measurements are performed. Subjects are asked about any side effects or AEs that may have occurred. Subjects receive additional packs of tablets, each containing 200 mg elemental calcium (a total of 100 tablets, 21 day supply+16 spare tablets) with the same formulation received in day 0. Subjects are instructed to take 4 tablets a day. Subjects that did not complete the distraction phase will be invited for an additional radiographic assessment based on the doctors' decision.

Visit 4—(Day 56±1)—Controlled antero posterior and lateral x-ray is performed. Functional assessment measurements are performed. Subjects are asked about any side effects or AEs that may have occurred. Subjects that did not show radiographic healing are invited for additional radiographic assessments based on the doctors' decision. Subjects receive additional packs of tablets, each containing 200 mg elemental calcium (a total of 100 tablets, 21 day supply+16 spare tablets) with the same formulation received in day 0. Subjects are instructed to take 4 tablets a day. Subjects that did not complete the distraction phase are invited for an additional radiographic assessment based on the doctors' decision.

Outcome Measures
Bone healing index (BHI)—bone healing index. A determination regarding the removal of the external fixator is made by identification of at least 3 of 4 cortical bridges in AP and lateral radiograph, in two manners:
   By 3 independent observers at each follow up evaluation.
   By using image analysis tools
Quantitative measurement of the distraction gap and callus calcification using image analysis tools
Consolidation time (CT)—the time (days) between the end of distraction osteotomy and total consolidation or removal of fixator
Functional assessments:
   Weight bearing
Assessment of symptoms and signs related to distal radius fractures:
Assessment of calcium side effects:
   Safety parameters: serum and urine calcium tests
   Adverse events recorded throughout the study.

Safety Analysis

The safety analyses are descriptive and narrative in nature. The safety endpoints are adverse events (AEs) and serious AEs (SAEs) whether or not related to study treatment. Also included are serum calcium levels and urine calcium and creatinine levels.

Efficacy Analysis

The primary efficacy endpoint is Bone Healing Index, by physician evaluation of radiographic imaging, scored semi-continuously based on the week in which healing is observed. “Time-to-Event” variables, such as the Bone Healing Index are described as continuous data (mean, median, etc.) since there is no censored data. To provide information on healing patterns of time, survival curves are constructed as well.

Hypotheses are tested by independent groups t-test on raw values if these are distributed approximately normal. If data deviate substantially from normal, log-transform is used to normalize the data. If after applying the latter the data still deviate from normal, the non-parametric Wilcoxon Rank-sum test is used.

The trial is considered successful if the Bone Healing Index as a function of the lengthening extent is significantly smaller in Treatment relative to Control.

The following are the study's secondary efficacy endpoints:
Bone Healing Index assessed by radiographic images analyzed by computerized image analysis.
Quantitative measurement of fracture gap and callus calcification, including:
   Time-to-Callus bridging fracture
   Time-to-Cortical bone crossing primary fracture line
   Amount and Density of New Bone Formation at the area of fracture gap
   The Amount and Density of Callus Formation
Change in Early Function

In addition to being presented as continuous variables, “Time-to-Event” endpoints such healing index by computerized image analysis are described by survival curves. Continuous endpoints such as Density of New Bone Formation are compared between groups using analysis of variance (ANOVA) or a non-parametric test if the data deviate meaningfully from normal. Missing data on continuous secondary endpoints is imputed using the last observation carried forward (LOCF) method. Change scores are computed using simple subtraction relative to baseline.

### Example 3

### Synthetic ACC for the Treatment of Distal Radius Fracture

Experiment aiming to evaluate the efficacy of administering synthetic stable amorphous calcium carbonate, comprising at least one stabilizer, for accelerating repair of bone fracture is conducted. A prospective, randomized, parallel, double-blind, controlled study compares the effect of amorphous calcium carbonate (ACC) versus either crystalline calcium carbonate (CCC) or placebo on functional outcome and radiographic healing time of distal radius fractures.

A fracture of the distal radius is one of the most common types of injuries to the skeletal system, and is treated using a variety of different techniques, from casting to pinning to open surgery with plates and screws. The Colles' fracture was chosen as the model to test the effects of ACC treatment on fracture healing because the distal radius includes both trabecular and cortical bone, is accessible for radiographs, has little soft tissue that can distort the radiograph, and is amenable to multiple functional endpoints. The primary objective of the trial is to assess the efficacy of treatment with calcium from ACC compared to CCC or placebo on radiographic healing time in subjects with distal radius fractures.

The secondary objective is to evaluate the effect of ACC compared to CCC or placebo on the improvement in wrist functional outcome following distal radius fracture. The further secondary objective is to evaluate the safety profile of ACC in this population.

Selection of Study Population

The study population includes 75 subjects with distal radius fractures, twenty five (25) subjects in each treatment group.

Inclusion Criteria:
Subjects with closed unilateral dorsally angulated fracture of the distal radius (Colles') visible by x-ray.
Subjects that can begin taking the study treatment exactly 7 (+1) days from the fracture event.
Subjects treated conservatively by closed reduction and immobilization.
Age 50-90 (inclusive).
Subjects able to adhere to the visit schedule and protocol requirements and be available to complete the study.
Subjects that had signed the ICF.

Exclusion Criteria:
Subjects with intra-articular fracture or extra-articular fracture that meets the criteria for operative fracture fixation.
Subjects with pins or plates in the wrist.
Sustained previous fractures or bone surgery in the currently fractured distal forearm
Subjects with multiple trauma (several fractures at once).
Subjects suffering from joint diseases that affect the function of the wrist and/or hand of the injured arm.
Elevated serum calcium (>10.2 mg/dL).
25-hydroxyvitamin D<20 ng/mL.
Subjects suffering from active liver disease or clinical jaundice.
Subjects with current or a history of a malignant neoplasm in the 5 years prior to the study.
Cognitive impairment.
Any disease affecting thyroid or parathyroid glands.
Chronic renal failure (Stage 3-5):
   Glomerular Filtration Rate (GFR)<60 ml/min
   Hyperphosphatemia, P>5 mg/dl.
Any known diseases affecting the absorption from the gastrointestinal tract:
   Inflammatory bowel disease (e.g., Crohn's disease, ulcerative colitis)
   Chronic diarrhea.

Description of the Investigational Product

The stable amorphous calcium carbonate used in the study is a synthetic ACC stabilized by low concentrations of phosphoserine and citrate (less than 0.5% in the final product), provided by Amorphical Ltd, as disclosed hereinabove.

Description of the Control Product

The efficacy of treatment of distal radius fracture with ACC was compared to the crystalline calcium carbonate and to placebo. The first control product contained in each tablet 500 mg of crystalline calcium carbonate (200±5 mg elemental calcium) and 167 mg of sucrose. Elemental calcium level in the control product was equal to the elemental calcium level in the treatment group in order to evaluate the effect of amorphous calcium carbonate compared to crystalline calcium carbonate. The second control product contained in each tablet 500 mg of starch. The healing of distal radius fracture, comprising administering calcium from the stable amorphous calcium carbonate source was compared to the healing with either crystalline calcium carbonate or placebo.

Dosage and Administration

Eligible subjects have randomly received one of the three treatments. Each dose of the study supplement consisted of 667 mg of ACC, containing 200 mg elemental calcium, or 500 mg of either CCC or starch in each tablet, as presented in table 5.

TABLE 5
Dose of Study Treatment

| Group | Treatment |
|---|---|
| Investigational | Tablets for oral use containing 667 mg ACC (200 |
| Product | mg elemental calcium) |
| Control Product 1 | Tablets for oral use containing 500 mg CCC (200 |
| | mg elemental calcium) and 167 mg of sucrose. |
| Control Product 2 | Tablets for oral use containing 500 mg starch |
| (placebo) | |

Selection of the dosage is based on the recommended daily intake of elemental calcium for subjects of age 19-50 (1000 mg/day). The calcium dosage in the control is equaled to the elemental calcium levels in ACC product.

Allocation of Subjects to Treatment

Subjects are assigned to one of the treatment groups randomly according to a randomization list. Randomization to each of the two study arms is performed using block randomization within center.

Blinding

The oral calcium treatments administered in the clinical trial are blinded. The subjects, the investigators and any personnel involved in subjects' assessment, monitoring, analysis and data management are blinded to the subject formulation assignment, except the Sponsor who is responsible for preparing, dispensing and labeling the investigational product. Blinded labels are affixed to the vials prior to dosing by the un-blinded Sponsor.

Randomization Procedures

The study is double blinded and therefore the CRC staff and the subject remain blinded to the code assignments throughout the study. Prior to administration, each subject is assigned with an individual number and is treated according to the predetermined computer generated randomization list. A computer-generated algorithm is used to assign the subject into the treatment groups. The treatment compositions are prepared by the Sponsor and labels are affixed to the vials prior to shipping. The hospital pharmacists are instructed to dispense the products to the CRC according to the cohort assignment lists.

Study Design

Seventy five (75) subjects are randomly assigned to one of three groups (N=25). Subjects in the treatment group receive amorphous calcium carbonate (ACC), those in the active control group receive the control product and those in the placebo group receive the placebo product. All formulations are supplemented with vitamin D. Safety parameters are evaluated throughout the trial.

Subjects admitting to the medical center hand clinic following a new distal radius fracture are routinely evaluated. Subjects who were initially treated according to current practice of fracture management, (i.e.; radiographic evaluation and fracture fixation by splint in plaster of Paris), who are not candidates for surgery and who are aimed for non-invasive treatment, are considered candidates for the trial and are invited to the CRC for screening.

Screening (Day −7)—Subjects sign an informed consent form (ICF). Chemistry and hematology tests are performed: sodium, potassium, hemoglobin, sedimentation rate, leukocytes calcium (total, albumin-corrected), phosphate, alkaline phosphatase, creatinine, and albumin. Also, serum PTH, 25-hydroxyvitamin D, and thyroid-stimulating hormone (TSH) are tested. Urinary excretion of calcium and creatinine is measured. General health is examined by medical history and physical examinations. Eligible subjects, complying with all inclusion criteria and none of the exclusion criteria are enrolled to the study.

Subjects are informed by phone or on site whether they are eligible to enter the study.

Visit 1—1 week from fracture—(Day 0 (−7/+1))—Eligible subjects are invited to the CRC. X-ray is performed to exclude loss of reduction. Subjects randomly receive packs of ACC or one of the control product tablets, wherein the investigational product tablet contains 200 mg elemental calcium (a total of 180 tablets, 42 day supply+12 spare tablets). Subjects who arrive prior to day 0 are instructed to begin the treatment exactly 7 days from the fracture event. Subjects are instructed to take 4 tablets a day for the first 6 weeks (days 0-42), 2 tablets in the morning and 2 tablets in the evening, after a meal (a total of 800mg elemental calcium per day in the investigational group). To minimize the risks for calcium related side effects, subjects in the investigational group and in the control group, wherein the control product is either CCC or placebo, who take calcium regularly, are instructed to discontinue their calcium supplements intake throughout the trial. Subjects are advised to take vitamin D3 supplementation based on the doctors' decision.

Visit 2—3 weeks from fracture—(Day 14±1)—Pre-dose serum calcium concentrations are measured. The cast is removed and controlled antero posterior and lateral x-ray is performed. Functional assessment (pain-free grip and force plate) measurement and questionnaires (VAS, DASH) are performed. The wrist is fixed using an adjustable brace. Subjects are asked about any side effects or AEs that may have occurred. Subjects complete the TSQM questionnaire with the representative of the CRC.

Visit 3—5 weeks from fracture—(Day 28±1)—Pre-dose serum calcium concentrations are measured. Controlled antero posterior and lateral x-ray is performed without the brace. Functional assessment (pain-free grip and force plate) measurement and questionnaires (VAS, DASH) are performed. Subjects are asked about any side effects or AEs that may have occurred.

Visit 4—7 weeks from fracture—(Day 42±1)—Pre-dose serum calcium concentration and urinary calcium and creatinine concentrations are measured. Controlled antero posterior and lateral x-ray is performed without the brace. Functional assessment (pain-free grip and force plate) measurement and questionnaires (VAS, DASH) are performed. Subjects receive additional packs of tablets, wherein the investigational product tablet contains 200 mg elemental calcium (a total of 180 tablets, 42 day supply+12 spare tablets) with the same formulation received on day 0. Subjects are instructed to take 4 tablets a day for the next 6 weeks (days 42-84), 2 tablets in the morning and 2 tablets in the evening, after a meal (a total of 800 mg elemental calcium per day in the investigational group). To minimize the risks for calcium related side effects, subjects in the investigational group and in the control group, wherein the control product is either CCC or placebo, who take calcium regularly, are instructed to discontinue their calcium supplements intake throughout the trial. Subjects are advised to take vitamin D3 supplementation based on the doctors' decision. Subjects who did not show radiographic healing in the x-ray are invited for an additional radiographic assessment on day 56.

Visit 5—9 weeks from fracture—(Day 56±1)—The tests are performed only for subjects that did not show radiographic healing on x-ray performed on previous visit. Controlled antero posterior and lateral x-ray is performed without the brace. Functional assessment (pain-free grip and force plate) measurement and questionnaires (VAS, DASH) are performed. Subjects are asked about any side effects or AEs that may have occurred. Subjects that did not show radiographic healing in the x-ray are invited for an additional radiographic assessment on day 70.

Visit 6—11 weeks from fracture—(Day 70±1)—The tests are performed only for subjects that did not show radiographic healing on x-ray performed on previous visit. Controlled antero posterior and lateral x-ray is performed without the brace. Functional assessment (pain-free grip and force plate) measurement and questionnaires (VAS, DASH) are performed. Subjects are asked about any side effects or AEs that may have occurred.

Visit 7—13 weeks from fracture—(Day 84±1)—Pre-dose serum calcium concentration and urinary calcium and creatinine concentrations are measured. Controlled antero posterior and lateral x-ray is taken. Functional assessment (pain-free grip and force plate) measurement and questionnaires (VAS, DASH) are performed. Subjects are asked about any side effects or AEs that may have occurred. Subjects complete the TSQM questionnaire with the representative of the CRC. The subjects are instructed to discontinue the study treatment administration. Subjects that discontinued their regularly calcium supplementation at the beginning of the study are instructed to continue their regular dose intake.

Visit 8—24 weeks from fracture—(Day 161±1)—Serum calcium concentration and urinary calcium and creatinine concentrations are measured. Controlled antero posterior and lateral x-ray is performed. Functional assessment (pain-free grip and force plate) measurement and questionnaires (VAS, DASH) are performed. Subjects are asked about any side effects or AEs that may have occurred.

Outcome Measures
Radiographic healing period—radiographic healing is defined as the interval in days between the occurrence of the fracture and the time when bridging in three of four cortices is seen on x-ray images. A determination is made for the two cortices (radial and ulnar) visible on the anteroposterior x-ray film and the two (dorsal and volar) seen on the lateral film in two manners:
   By 3 independent observers at each follow up evaluation.
   By using image analysis tools
Quantitative measurement of fracture gap and callus calcification using image analysis tools
Functional assessments:
   Pain-free grip—assessment of grip strength via a JAMAR dynamometer.

The dynamometer is linked to software that easily and accurately measures grip strength. The dynamometer measures in increments of 0.1 kg. The mean of the three measurements, 2 min apart, will be considered as the grip strength for a patient at a specific visit. To adjust for hand dominance in grip strength, if the non-dominant hand was injured, the percentage is multiplied by 1.07; if the dominant hand was injured, the percentage will be multiplied by 0.93.
Pain free weight bearing—assessment of hand weight bearing is measured using FP2 force plate. The FP2 force plate is controlled by the amount of force applied and can be set in 0.1 kg increments. The force plate is very sensitive and responds to as little as the touch of a finger. The maximum force generated is measured by software connected to the force plate.
Assessment of symptoms and signs related to distal radius fractures:
   DASH score—a 30-item, self-report questionnaire designed to measure physical function and symptoms in people with any of several musculoskeletal disorders of the upper limb.
   Pain evaluation by VAS questionnaires.
Assessment of calcium side effects:
   Safety parameters: serum and urine calcium tests
   Treatment satisfaction questionnaire for medication (TSQM).
Adverse events recorded throughout the study.

Safety Analysis

The safety analysis is descriptive and narrative in nature. The safety endpoints are adverse events (AEs) and serious AEs (SAEs) whether or not related to study treatment. Also included are serum calcium levels and urine calcium and creatinine levels and the side effects domain of the Treatment Satisfaction Questionnaire for Medication (TSQM).

Efficacy Analysis

The primary efficacy endpoint is Time-to-Healing, by physician evaluation of radiographic imaging, scored semi-continuously based on the week in which Healing is observed. “Time-to-Event” variables, such as Time-to-Healing are described as continuous data (mean, median, etc.) since there is no censored data. To provide information on healing patterns of time, survival curves are constructed as well.

Hypotheses are tested by independent groups t-test on raw values if these are distributed approximately normal. If data deviate substantially from normal, log-transform is used to normalize the data. If after applying the latter the data still deviate from normal, the non-parametric Wilcoxon Rank-sum test is used.

The trial is considered successful if Time-to-Healing is significantly shorter in Treatment relative to Control.

The following are the study's secondary efficacy endpoints:
Time-to-Healing assessed by radiographic images analyzed by computerized image analysis.
Quantitative measurement of fracture gap and callus calcification, including:
   Time-to-Callus bridging fracture
   Time-to-Cortical bone crossing primary fracture line
   Amount and Density of New Bone Formation at the area of fracture gap at 3, 5, 7 and 13 weeks from fracture
   The Amount and Density of Callus Formation at 3 ,5, 7 and 13 weeks from fracture
Change in Early Function (pain-free grip and force plate measurements)
Change in symptoms and signs related to distal radius fractures: DASH and VAS
TSQM domains: Effectiveness, Convenience and Global Satisfaction

In addition to being presented as continuous variables, “Time-to-Event” endpoints such Time-to-Healing by computerized image analysis are described by survival curves. Continuous endpoints such as Density of New Bone Formation are compared between groups using analysis of variance (ANOVA) or a non-parametric test if the data deviate meaningfully from normal. Missing data on continuous secondary endpoints is imputed using the last observation carried forward (LOCF) method. Change scores are computed using simple subtraction relative to baseline.

### Example 4

### Synthetic ACC for the Acceleration of Dental Implants Integration

Dental implants are surgically inserted in the tibia of rabbits and are allowed to heal for 2 to 6 weeks. The rabbits are orally administered with a single gelatin capsule containing either synthetic ACC or CCC (crystalline calcium carbonate). Following the healing intervals, animals are sacrificed and the tibia are harvested. Some of the implants are removed for scanning electron microscopy (SEM).

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1-35. (canceled)

36. A method of accelerating bone growth in a subject having a bone condition selected from the group consisting of a distraction osteogenesis, osseointegration, fracture by external force, pathological fracture, fatigue fracture, osteotomy, and combinations thereof, the method comprising:
orally administering to the subject an effective amount of a composition including stable amorphous calcium carbonate (ACC) having at least one stabilizer.

37. The method according to claim 36, wherein the method provides from about 0.5 to about 1.5 mm/day bone growth rate.

38. The method according to claim 36, wherein the method further provides enhancement of a functional outcome following the bone condition.

39. The method according to claim 36, wherein the bone condition includes a distraction osteogenesis procedure.

40. The method according to claim 39, wherein the composition is administered during a latency phase of the distraction osteogenesis procedure, a distraction phase of the distraction osteogenesis procedure, a consolidation phase of the distraction osteogenesis procedure, or any combination thereof.

41. The method according to claim 39, wherein the composition is administered starting at least two days from a surgery of the distraction osteogenesis procedure.

42. The method according to claim 39, further comprising accelerating bone consolidation following bone distraction.

43. The method according to claim 36, wherein the bone condition includes an osseointegration procedure.

44. The method according to claim 43, wherein the composition is administered following an implant insertion stage of the osseointegration procedure, following an abutment insertion stage of the osseointegration procedure, or a combination thereof.

45. The method according to claim 43, wherein the osseointegration procedure includes a dental implant integration.

46. The method according to claim 36, wherein the pathological fracture includes a fracture associated with osteoporosis, osteomalacia, malignant tumor, multiple myeloma, osteogenesis imperfecta congenita, cystic bone, suppurative myelitis, osteopetrosis, nutrition disorders, or a combination thereof.

47. The method according to claim 36, wherein the bone fracture by external force includes a non-union fracture, a mal-union fracture, or a delayed union fracture.

48. The method according to claim 36, wherein the composition is administered at least once a day.

49. The method according to claim 36, wherein the composition is administered at a daily dose of about 0.5 to about 5 g ACC.

50. The method according to claim 36, wherein the composition is administered in combination with at least one vitamin selected from the group consisting of Vitamin C, Vitamin D, Vitamin E, and Vitamin K.

51. The method according to claim 36, wherein the at least one stabilizer is selected from the group consisting of organic acids, phosphorylated organic acids, phosphoric or sulfuric esters of hydroxy carboxylic acids, phosphorylated amino acids and derivatives thereof, hydroxyl bearing organic compounds, and combinations thereof.

52. The method according to claim 51, wherein the phosphorylated amino acids are present in oligopeptides or polypeptides.

53. The method according to claim 51, wherein the phosphorylated amino acids are selected from the group consisting of phosphoserine and phosphothreonine.

54. The method according to claim 51 wherein the at least one stabilizer includes a hydroxyl bearing organic compound selected from the group consisting of mono-, di-, tri-, oligo-, and polysaccharides.

55. The method according to claim 54, wherein the at least one stabilizer includes a carboxylic acid.
